(19) United States
(12) Patent Application Publication
(10) Pub. No.: US 2013/0169971 A1
(43) Pub. Date: Jul. 4, 2013

(54) OCT IMAGING SYSTEM FOR CURVED SAMPLES

(71) Applicant: Wasatch Photonics, Inc., Logan, UT (US)

(72) Inventors: William J. Brown, Durham, NC (US); Michael E. Sullivan, Raleigh, NC (US); Scott M. Norton, Durham, NC (US)

(73) Assignee: WASATCH PHOTONICS, INC., Logan, UT (US)

(21) Appl. No.: 13/684,176

(22) Filed: Nov. 22, 2012

Related U.S. Application Data
(60) Provisional application No. 61/563,241, filed on Nov. 23, 2011.

(51) Int. Cl. A61B 3/10

(52) U.S. Cl. CPC A61B 3/102
USPC 356/479

(57) ABSTRACT

This patent specification describes an OCT imaging system that implements at least one of the following: (1) adjusting the path length of a reference arm during an OCT scan of a curved sample or between OCT scans; (2) adjusting the focus of a scanning beam as the scanning beam moves across the curved sample during the OCT scan or between OCT scans; (3) adjusting polarization control during the OCT scan or between OCT scans; and/or (4) changing dispersion compensation across the OCT scan or between OCT scans.

### BACKGROUND

Optical coherence tomography (OCT) was first developed in the early 1990s and is now an established imaging modality with numerous medical applications and some uses in industry. The primary market remains retinal imaging of the human eye and there are at least eight companies offering products for this application. The technological implementation of OCT has evolved from early time-domain systems to Fourier domain systems, using both spectral domain (broadband source with spectrometer) and swept source (swept laser source with photodiode detector). Current systems typically have an imaging depth in tissue of 1 to 2 millimeters, although a few can reach 5 or 6 millimeters for anterior segment imaging and other applications.

### SUMMARY

This patent specification describes an OCT imaging system that implements at least one of the following: (1) adjusting the path length of a reference arm during an OCT scan of a curved sample or between OCT scans; (2) adjusting the focus of a scanning beam as the scanning beam moves across the curved sample during the OCT scan or between OCT scans; (3) adjusting polarization control during the OCT scan or between OCT scans; and/or (4) changing dispersion compensation across the OCT scan or between OCT scans.

### BRIEF DESCRIPTION OF THE DRAWINGS

FIGS. 1A and 1B illustrate an OCT system.

FIG. 2 shows a typical image of the retina of a human eye taken with an OCT system.

FIG. 3 illustrates an OCT image of the cornea and anterior chamber of a human eye.

FIG. 4 illustrates some of the terminology that is used throughout the present patent specification.

FIG. 5 illustrates an OCT engine that includes one possible implementation of a reference arm path length adjustment.

FIGS. 6A and 6B illustrate one possible implementation of a rapid scanning optical delay (RSOD).

FIG. 7 illustrates an OCT scan head that includes one possible implementation of a mechanism for adjusting the focus of a scanning beam.

FIG. 8 illustrates an OCT scan head that includes another possible implementation of a mechanism for adjusting the focus of a scanning beam.

FIG. 9 illustrates an OCT engine that includes one possible implementation of a mechanism for adjusting polarization control.

### DETAILED DESCRIPTION

This patent specification describes an OCT imaging system capable of imaging curved samples, such as the retina, cornea, plastic tubes, and others.

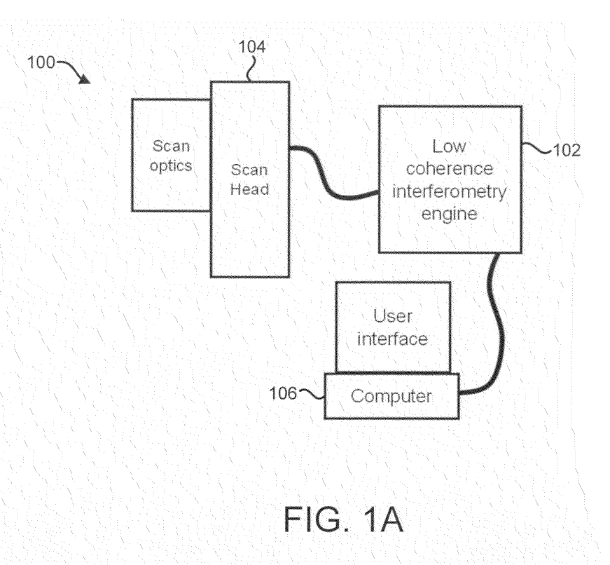

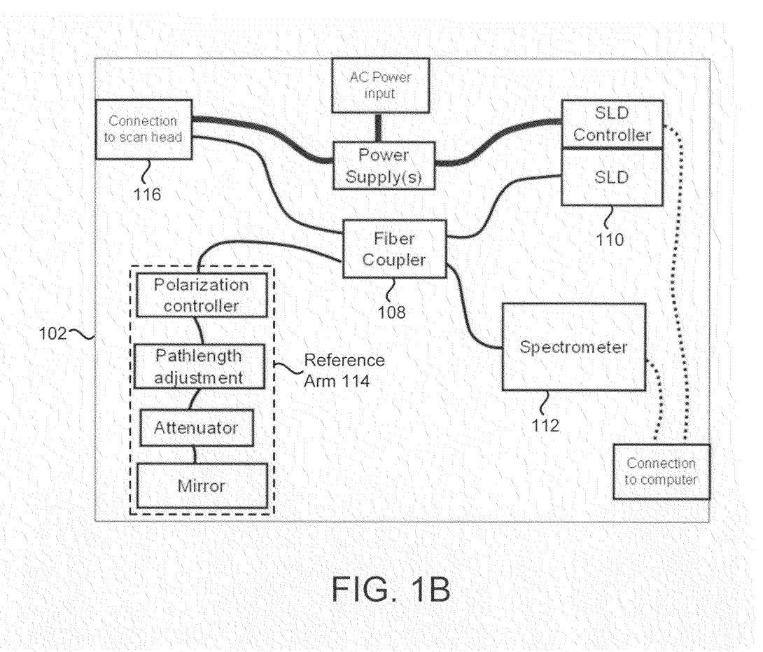

FIG. 1A is a system block diagram of an OCT system 100 with a low coherence interferometry engine 102 (which may also be referred to as an OCT engine 102), a scan head 104 and a computer 106 with software. FIG. 1B is a block diagram showing some details of the OCT engine 102, including an interferometer (fiber coupler 108), light source (superluminescent laser diode 110), spectrometer 112, reference arm 114, and a connection 116 out to the sample arm in the scan head 104.

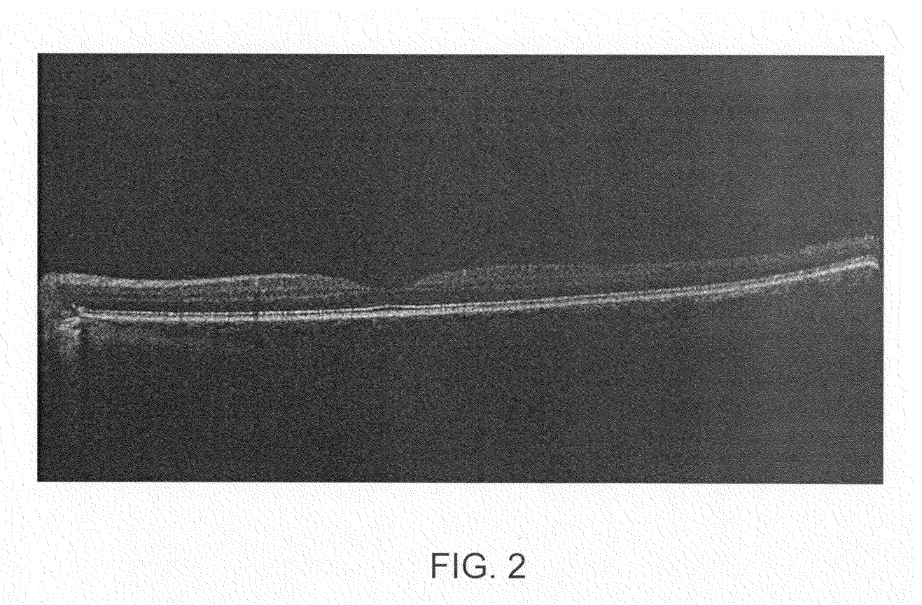

FIG. 2 shows a typical image of the retina of a human eye taken with an OCT system (such as the OCT system 100 shown in FIGS. 1A and 1B). On the left is part of the optical nerve and near the middle is fovea, which shows up as a dip in the scan. Farther to the right, the image begins to curve upward due to the different curvatures of the OCT scan and the retina. The OCT scan typically pivots around the pupil at the front of the eye, whereas the retina is on a circle centered at the middle of the eye. For a scan of up to 6 or 8 millimeters in length, the variation in path length is less than the imaging depth of the OCT system and the image remains in the depth of field. However for longer scans the path length variation is significantly greater and will take the image outside of the depth of field of the OCT system.

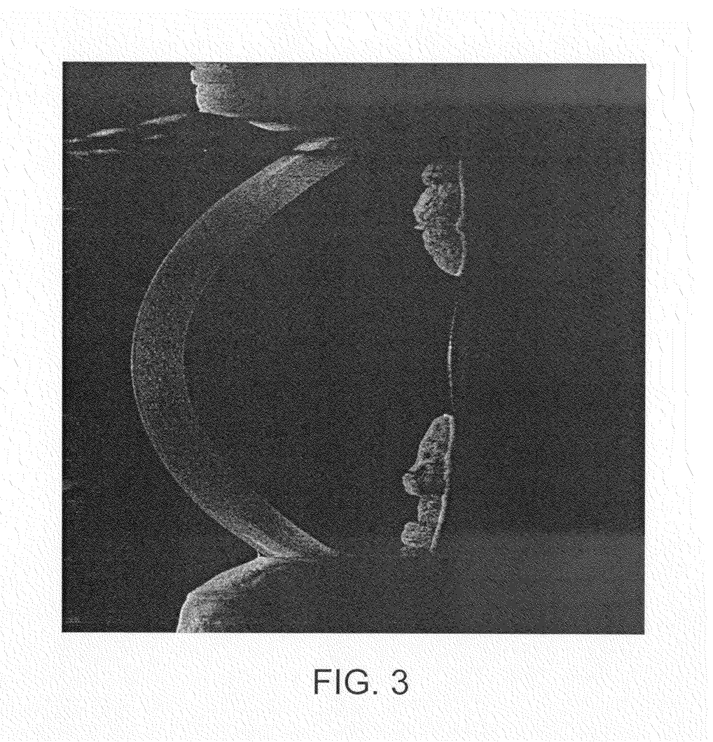

Another sample with a curved surface is the cornea at the front of the human eye. U.S. patent application Ser. No. 13/267,574 (hereinafter, “the '574 application”) covers a deep imaging OCT system, particularly for use in imaging the anterior chamber of a human eye. FIG. 3 illustrates an OCT image of the cornea and anterior chamber of a human eye taken with a system covered by the '574 application. This scan is vertical with the subject facing to the left. The cornea is clearly visible on the left (the curved surface is the cornea), and the iris and the front surface of the lens are visible on the right (the iris is on the top and bottom, and the front surface of the lens is in between). The “flashes” at the top are eye lashes in front of the eyelid.

Imaging the cornea is one example of a curved field scan. Even though the cornea is ˜1 millimeter thick, the curvature results in an imaging range of 4 or 5 millimeters.

In addition to these examples, there are many other OCT imaging applications where the imaging range is much larger than the actual imaging depth. Similarly, in the retina, as OCT is scanned into the periphery, the curvature of the eye relative to the pupil results in a shorter path length which may degrade the image quality or be completely out of the range of the system.

This patent specification provides a solution to imaging samples where significant imaging depth is required.

There are four pieces to improving the imaging depth of the OCT system. It is not necessary that all four pieces be implemented in a particular system. The four pieces are:

1) reference arm path length that adjusts during an OCT scan or between OCT scans;

2) adjustable focus of the OCT beam on the sample during an OCT scan or between OCT scans;

3) polarization control that changes during an OCT scan or between OCT scans; and

4) dispersion compensation that varies across the OCT scan or between OCT scans.

Varying the dispersion compensation across the OCT scan may comprise changing the dispersion compensation as the scanning beam moves laterally across the curved sample. Alternatively, varying the dispersion compensation across the OCT scan may comprise optimizing the dispersion compensation as a post-processing step.

### TERMINOLOGY USED

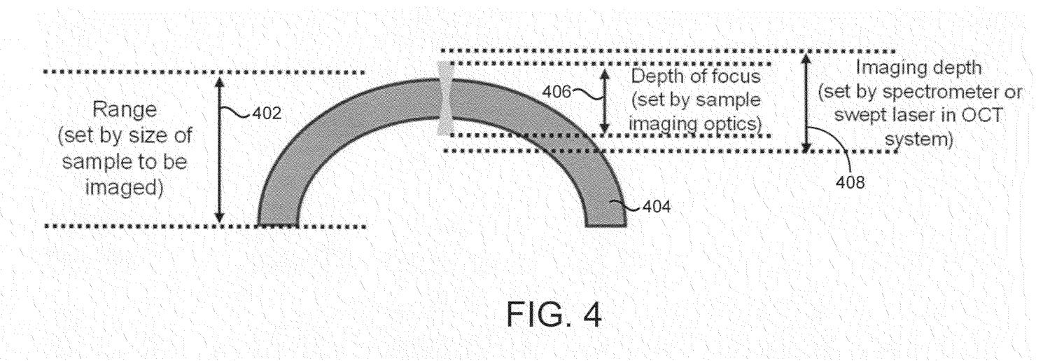

FIG. 4 illustrates some of the terminology that is used throughout the present patent specification. Range 402 refers to the entire depth of the sample 404 that is to be imaged by the OCT system. Depth of focus 406 refers to the range over which the spot size is within a factor of 2 of the minimum spot size (or waist). Imaging depth 408 refers to the depth over which the OCT system can image in a single A-scan and is set by either the coherence length of one pixel in the spectrometer or the wavelength range covered by a swept source laser in one integration period.

As used herein, the term “A-scan” refers to a one-dimensional sample reflectivity profile acquired along a depth direction. The term “B-scan” refers to a two-dimensional set of image data that is generated by collecting A-scans at adjacent transverse positions. The term “C-scan” refers to a three-dimensional scan, in which the scan mirror or mirrors are scanned in more than one axis. The term “OCT scan” may refer to an A-scan, a B-scan, or a C-scan.

Adjustable Reference Arm Path Length

In order to keep the sample within the imaging depth of the OCT system as the beam is scanned laterally, the reference arm path length can be changed.

Unlike time domain OCT where the reference arm path length may be changed at high speed to generate the A-scan at an individual location, in spectral domain OCT the path length may be changed at a relatively slow speed over the course of the B-scan (or area scan). These changes could be synchronized with the A-scan acquisition to reduce reference arm motion during an acquisition or they may follow a predefined profile.

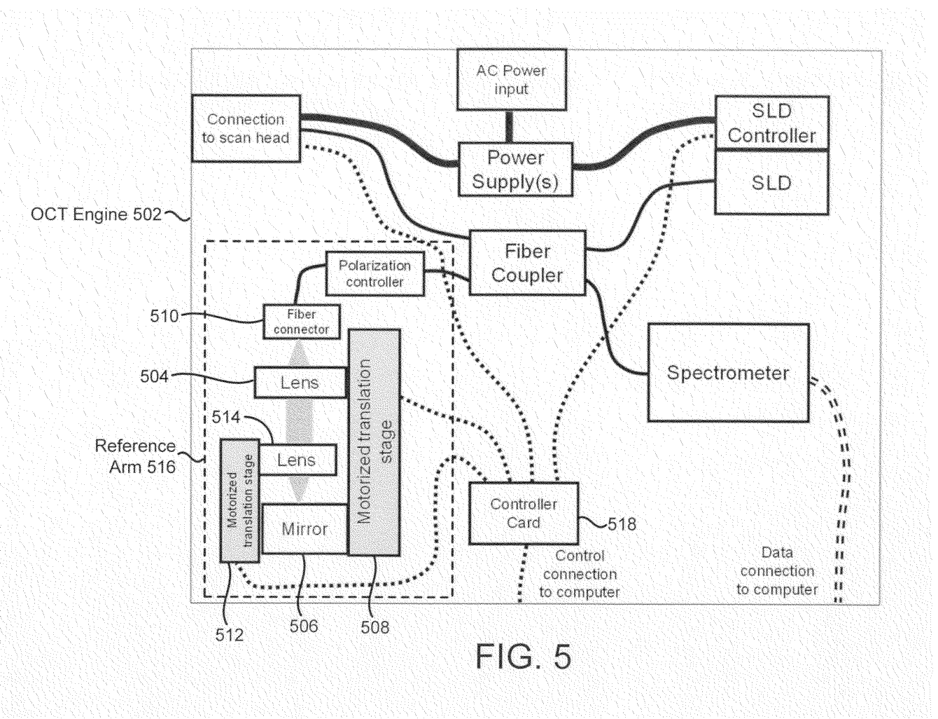

There are several ways to implement the reference arm path length adjustment. FIG. 5 is a block diagram of an OCT engine 502 that includes one possible implementation where a first focusing lens 504 and a mirror 506 may be mounted on a motorized translation stage 508. The motorized translation stage 508 may move the first focusing lens 504 and the mirror 506 relative to a fiber connector 510. A second motorized translation stage 512 may move a second focusing lens 514 relative to the mirror 506, thereby providing adjustable optical attenuation as the distance between the second focusing lens 514 and the mirror 506 moves in and out of the focal length of the second focusing lens 514.

Alternately, the optical power control can be implemented using a liquid lens with or without an additional focusing lens. By changing the focal length of the liquid lens, the focus is changed resulting in a change in the optical throughput in a similar fashion to moving the lens relative to the mirror. This implementation may be lighter, faster and cheaper than using a motor and a translation stage.

In order to increase the path length of the reference arm 516, the motorized translation stage 508 may move the first focusing lens 504 and the mirror 506 farther away from the fiber connector 510. Conversely, in order to decrease the path length of the reference arm 516, the motorized translation stage 508 may move the first focusing lens 504 and the mirror 506 closer to the fiber connector 510. During an OCT scan, the path length may be increased or decreased, as appropriate, in order to keep the sample within the imaging depth of the OCT imaging system.

The motorized translation stages 508, 512 may be controlled by a controller card 518 (with onboard firmware) in the OCT engine 502. The controller card 518 may be controlled by software on a computer (e.g., a computer similar to the computer 106 shown in FIG. 1A). Alternatively, the computer may directly control the motorized translation stages 508, 512 without passing through an intermediate control card.

Design for the motorized translation stages 508, 512 will depend on the speed needed and the weight of the optics and mechanics that are on the stages 508, 512. As an example, for cornea imaging, the reference arm 516 may need to move back and forth (one full cycle) by 3 to 6 millimeters within the time of one scan. There is a wide range of OCT system imaging speeds (typically referred to as A-scan rate) used. For example, one Wasatch Photonics system is capable of 45,000 lines/second (or A-scans per second). A B-scan can be composed of a wide range of A-scans, but typically are within the range 200 to 4000. Assuming 2,000 A-scans in a B-scan and 45,000 A-scans per second, then one image will be acquired in approximately 2/45 seconds or 44 milliseconds. Covering a full cycle of 6 millimeters gives a total travel range of 12 millimeters in 45 milliseconds or 266 millimeters per second. This is fairly fast, but within the range of currently available motorized stages. Since the typical path is fairly smooth (i.e., no stops and starts except at the beginning and end of the travel), the acceleration and hence the forces required are minimal.

Another possible implementation is to use a rapid scanning optical delay (RSOD) in the reference arm. RSODs are often used in time domain systems. RSODs are designed to cover the full imaging depth of the OCT system with up to 5,000 scans per second. For this implementation, the number of cycles per second would match the number of B-scans per second and would be in the range of a few up to 40 or 50 cycles per second.

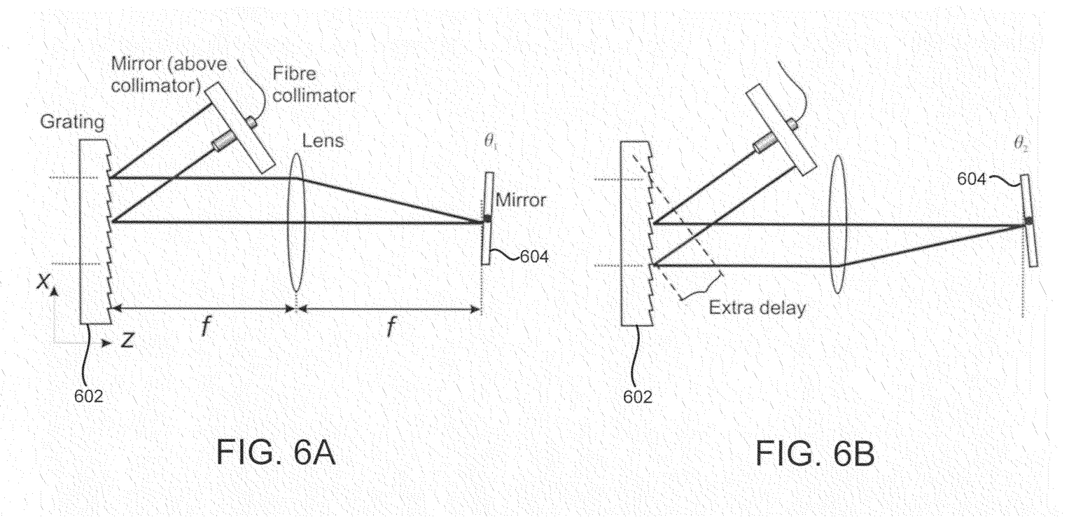

See FIGS. 6A and 6B for one possible implementation of an RSOD 602 based on a reflective diffraction grating 604 and a rotating galvo mirror 606. The implementation shown in FIGS. 6A and 6B is taken from the website of The Optical+Biomedical Engineering Laboratory in the School of Electrical, Electronic and Computer Engineering at the University of Western Australia in Perth, Western Australia, which is currently accessible via the World Wide Web at http://obel.ee.uwa.edu.au/research/engineering/fdodl/.

In the RSOD 602 the light exits the fiber collimator 608, is diffracted off of the grating 604, passes through the lens 610, reflects off of the mirror 606, passes back through the lens 610, diffracts off of the grating 604 and hits a mirror 612 above the fiber collimator 608. The light is reflected off of this mirror 612 and retraces its path back to the fiber collimator 608. By changing the angle of the mirror 606, the location where the beam hits on the grating 604 for the second time is changed. This results in a change in the path length traced out by the light from the exit of the fiber collimator 608 until it returns to the fiber collimator 608.

In one implementation of the present invention, an RSOD (such as the RSOD 602 shown in FIGS. 6A and 6B) may be used to provide the same functionality as the motorized stage 508 in the reference arm 516 in the OCT engine 502 shown in FIG. 5. The RSOD may then be used with an optical power control device and polarization controller to provide the same functionality as in FIG. 5.

Unlike time domain OCT, where the motion of the stage generates the fringes that constitute the OCT signal, in this case motion of the reference arm may blur the fringes that occur as a function of wavelength across the spectrometer or the sweep of a tunable laser. There are several potential solutions to this issue. The first is that the scan may be slow enough that it does not significantly affect the fringes. If 2,000 A-scans are acquired as the reference arm moves 12 millimeters (the same parameters as used to calculate translation speed) then the motion per A-scan is only 6 microns. This is on the order of the axial resolution of typical OCT systems, so the amount of blurring may not severely affect the signal level. If it is an issue, decreasing the scan range or increasing the A-scan rate will reduce the impact.

Alternatively, the reference arm could be designed to have the majority of the motion occur between the A-scan acquisitions. That is, the system would acquire an A-scan, then move the reference arm, and then acquire the next A-scan. There could be some overlap between the motion of the reference arm and the A-scan acquisition. This would require higher acceleration and forces from the motor that drives the reference arm since it would be starting and stopping the motion of the stage.

These are just a few of the possible hardware implementations of a scanning reference arm. Others will be apparent to those skilled in the art. The important concept is that the reference arm scans in path length while the OCT system scans laterally across the sample and there is coordination between the two scans.

Adjustable Sample Arm Focus

The next piece is an adjustable focus of the scanning beam as it moves across the sample. Since lateral resolution and scattered signal strength increase as the spot size is decreased, OCT systems are typically designed with the smallest possible spot size. However, the smaller the spot size, the shorter the depth of focus (sometimes referred to as depth of field). The depth of focus is given by b=2*π*ω²/λ, where b is the depth of focus, ω is the beam waist (or spot size), and λ is the wavelength of the light. As an example in the retina where the typical spot size is 20 microns and the wavelength of light is 840 nm, the depth of focus is 2.9 millimeters.

There are several possible hardware implementations of this concept. The first is to use a liquid lens. Liquid lenses are a recent development. One company that currently provides liquid lenses is Edmunds Optics. The Edmunds Optics website states the following about liquid lenses (which it calls electrically focus-tunable lenses): “Electrically focus-tunable lenses provide a single-lens solution for focus and zoom objectives. When the user applies a voltage to each compact, plano-convex lens, the lens changes effective focal length. Each lens is filled with a proprietary optical liquid, and the change in voltage alters the pressure profile of the liquid, resulting in a change in radius of curvature. This electrical manipulation of the radius allows for a focal range of +15 to +100 mm and, along with an aperture of 10 mm, provides an impressive variety of applications and can replace multiple elements in an optical system. These robust lenses are available in high refractive index or low dispersion options for research and for OEM integration. Cover glasses for the electrically focus-tunable lenses are available with either a VIS or NIR coating.”

One potential liquid lens that may be used to provide an adjustable focus of the scanning beam as it moves across the sample is the Edmund Optics NT83-922, which has a clear aperture of 10.0 millimeters, a focus range of +45 mm to +120 mm and is coated for the NIR (near infrared, ˜500 nm to ˜1100 nm). This lens is manufactured by Optotune and is distributed by Edmund Optics. Another lens is the Artic brand liquid lens from Varioptic.

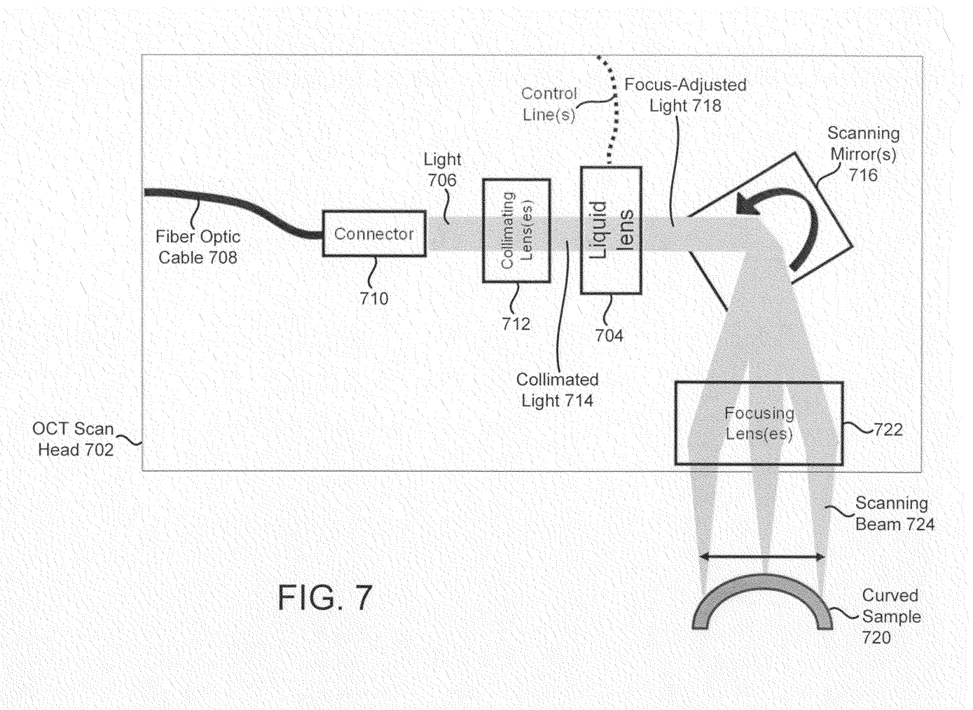

FIG. 7 shows one possible block diagram for an OCT scan head 702 using a liquid lens 704. Light 706 enters the OCT scan head 702 via a fiber optic cable 708. The light 706 exits the fiber optic cable 708 at a connector 710 and is collimated by one or more collimating lenses 712. Next, collimated light 714 passes through the liquid lens 704. The focus of the collimated light 714 is dynamically adjusted by the liquid lens 704. In other words, the liquid lens 704 may impart some convergence (focusing) or divergence (defocusing) to the collimated light 714. One or more scanning mirrors 716 are used to scan the focus-adjusted light 718 across a sample 720 via one or more focusing lenses 722. More specifically, the focus-adjusted light 718 is scanned laterally by the scanning mirror(s) 716. The focusing lens(es) 722 focus the scanning beam 724 on the sample 720. The liquid lens 704 allows the focal point of the scanning beam 724 (i.e., the spot with the smallest beam waist) to be varied in depth as the scanning beam 724 is scanned laterally. In an alternative implementation, the liquid lens 704 may be in front of the collimating lens(es) 712.

Another implementation is to move the collimating lens(es) relative to the input fiber connector. This will result in a beam that is either slightly diverging or converging, which after passing through the focusing lens(es) will give a beam waist that is either slightly farther away than the focal length or slightly closer. As long as the adjustment is minor, the beam waist will only change by a small amount. Typically the focusing lens(es) will have a focal length in the range of 25 to 100 millimeters, so a shift of up to 5 millimeters is a small fraction of the overall focal range.

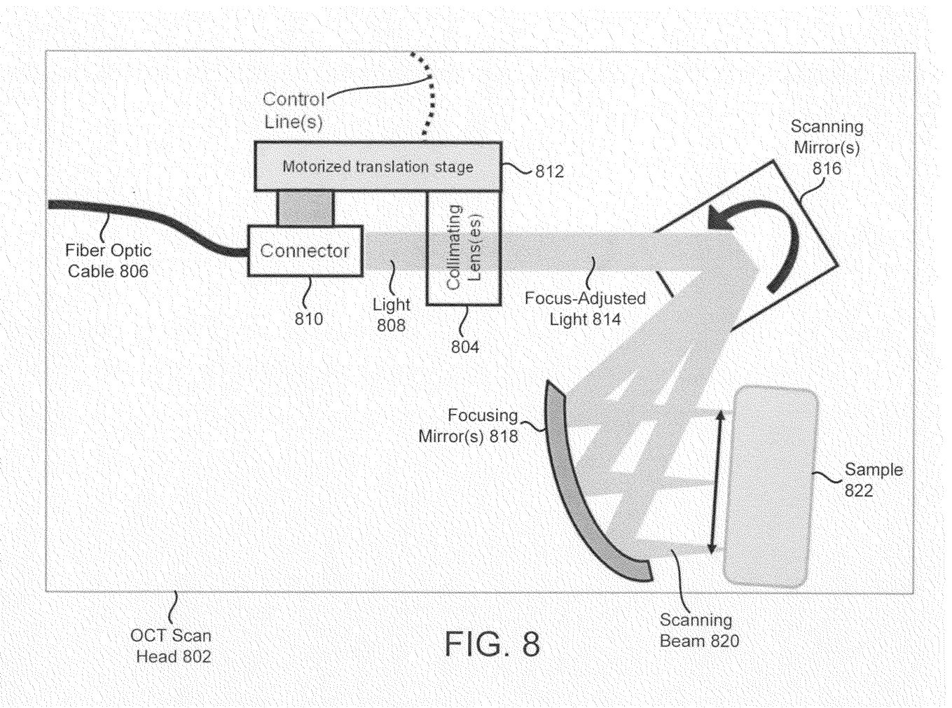

FIG. 8 shows one possible implementation of an OCT scan head 802 where one or more collimating lenses 804 after the input fiber optic cable 806 are moved relative to the input fiber optic cable 806 to impart some focusing or defocusing to the beam. Again, the light 808 enters via the fiber optic cable 806 and exits at a connector 810. A motorized translation stage 812 moves the collimating lens(es) 804 slightly relative to the connector 810 so that focus-adjusted light 814 exiting the collimating lens(es) 804 may be slightly divergent, collimated or slightly focusing. One or more scanning mirrors 816 move the focus-adjusted light 814 laterally. One or more focusing mirrors 818 focus the scanning beam 820 on the sample 822. The scanning beam 820 is again scanned across the sample 822 with a beam waist that moves in depths as the scanning beam 820 is scanned.

Note that in these diagrams the collimating and focusing optics are shown as transmissive optics, most likely made from glass. This design also works when some or all of the optics are reflective optics (typically mirrors such as aluminum, silver, or gold-coated mirrors, or dielectric mirrors).

Adjustable Polarization Control

Polarization control refers to the changes made to the polarization optics in the sample or reference arm to match the polarizations, and hence the interference signal, as much as possible. Polarization effects may arise from the fiber components, the free space optics, and/or the sample itself.

Polarization control (or correction) may be accomplished in many ways. One way is to use loops of fiber that are rotated about one side of the loop. A setup with two loops, one with 1 coil of fiber and one with 2 coils of fiber, approximates a quarter waveplate and a half waveplate. Rotation of these two fiber coils allows the majority of the possible polarization states to be covered. Since the interference signal goes as the dot product of the reference and signal arm light, it is important that the polarizations be close to each other, but it is not necessary that they be exactly the same.

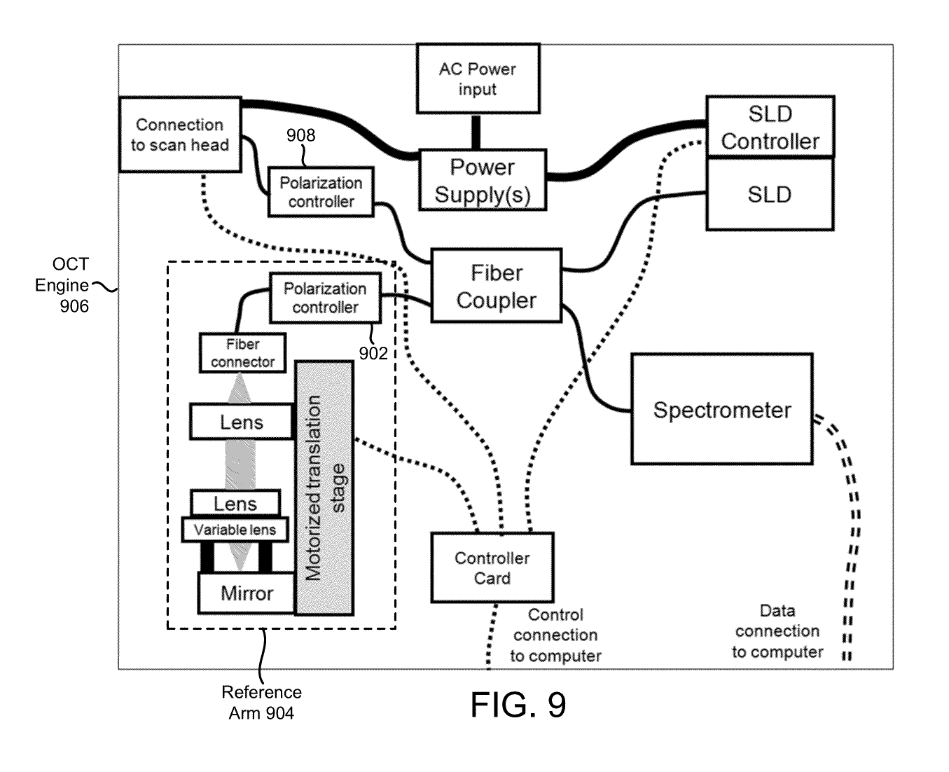

The polarization control may be in the reference arm or in the sample arm or both. There are advantages to different configurations. FIG. 9 illustrates one implementation in which a polarization controller 902 is included in the reference arm 904, and another polarization controller 908 is included in the sample arm. The polarization controller 902 in the reference arm 904 may be optimized at system setup or startup. The polarization controller 908 in the sample arm may then be adjusted to compensate for changes in the sample or the sample arm. For example, if the fiber between the OCT engine 906 and the scan head changes position, resulting in a change in polarization, the polarization controller 908 in the scan arm may then be adjusted to compensate for this change. One advantage of this approach is that the reference arm 904 does not need to change as the sample changes.

Variable Dispersion Compensation

Dispersion compensation refers to adjustments made to the wavelength or wavenumber of the pixels in the spectrometer to account for the fact that different wavelengths of light experience different indices of refraction (and hence optical path lengths) in the reference and sample arm. Mismatched dispersion (i.e., dispersion that only happens in one arm of the interferometer and not both) must be compensated for or the OCT image will be blurred in the axial direction. Historically, dispersion compensation was often accomplished by adding physical dispersion (such as a piece of glass) to either the sample or the reference arm of the interferometer. It is still the case that gross dispersion compensation may be done by physical means, but most dispersion compensation is now performed in software or firmware.

This dispersion compensation may either be generated by physically measuring the dispersion and then adding software dispersion compensation or just by optimizing the image as a function of the dispersion parameters.

For curved samples it may be necessary to change the dispersion compensation as the beam is laterally moved across the sample. This is more in cases where the sample sits behind some dispersive material. For example, the retina is behind the water in the eyeball. This varying dispersion may be sufficient to blur the image for path lengths that do not match the path length where the dispersion was optimized. In this case the dispersion compensation can be varied across the lateral scan to maintain a high level of OCT signal across the entire scan.

Typically in Fourier Domain OCT systems the dispersion compensation consists of two or three correction terms which are introduced after the wavelength data is resampled to wavenumber. The first correction term is quadratic (since the linear term just changes the frequency of the entire fringe) and the 2nd term is tertiary. Occasionally a 4th order term may be used. Adjusting these two or three correction terms (or parameters) across the scan may be sufficient to maintain a high level of OCT signal since a polynomial described by two or three parameters should be a good fit for any variations in dispersion across the wavelength ranges and optical path length ranges that occur in OCT systems. Other dispersion compensation techniques use higher order polynomials or other basis functions to create a correction function. Empirical data can also be used.

Dispersion correction can be automated based on some criteria such as maximizing the variance of the A-scan. This can be done on a windowed subset of the OCT scan. For example, the dispersion correction values for a particular A-scan could be based on the 5 A-scans centered at that particular A-scan. The window would then be moved across the entire B-scan generating locally optimized dispersion compensation values. The window size can vary from 1 A-scan up to the entire number of A-scans in the B-scan.

Dispersion compensation may occur in real time as the B-scan is acquired, or it may be post processed after one or more B-scans have been acquired. In another implementation, coarse correction values could be used with automated generation of a correction value again either in real time or in post processing.

Required Pieces

It is not necessary to have all four pieces in a given OCT imaging system. For example, if imaging the cornea, the variable dispersion compensation may not be needed since the path length variation occurs in air and air has relatively low dispersion. Likewise if the depth of focus of scanning optics is sufficient, the variable focus length lens may not be needed.

2D and 3D

The description and the sample images provided have been primarily B-scans, or 2D images where A-scans (1D depth profiles) are acquired as the beam is scanned across the sample in one direction. Everything described can also be used to acquire 3D images where the beam is scanned in two dimensions across the sample. For the cornea, the OCT image could be of the entire cornea and made of a set of horizontal B-scans, vertical B-scans, radial B-scans or some other scan patterns. Likewise for the retina, this may enable imaging across the entire back of the eye (180 degrees or more measured from the center of the eye) in both the horizontal and vertical direction.

System Architectures

There are at least two general approaches to the software control of the ranging adjustments: a pre-set scan profile that is loaded or calculated from memory or storage, or a scan profile that is adjusted during a scan and/or between scans based on information in the image. Either approach may be used independently for each of the four adjustments, (1) path length, (2) focus, (3) polarization control and (4) dispersion compensation.

1. Pre-Set Scan Profiles

In this approach the system software and/or firmware adjusts the path length, focus, and/or dispersion compensation during an OCT scan based on a profile that is loaded or calculated from memory or other location. This profile is then static for that particular image (or images). More specifically, the pre-set scan profile is static during the OCT scan that generates the image(s).

As an example, the path length profile for imaging the cornea may follow a parabola with the shortest path length in the middle of the scan and the path length on each end longer by several millimeters. The points in between may fall on the parabola defined by the endpoints and the middle point.

As another example, the focus could be shifted based on the expected variation over the scan. This could be used in wide-field retinal imaging to maintain a small spot size on the retina in the periphery where the retina is closer to the pupil than at the fovea.

2. Real-Time Profiles Based on Sample

In this approach, the system software and/or firmware may start from a known profile for the path length, focus, or dispersion and the profile may be modified based on information in an OCT image. This may be done on an A-scan basis or between B-scans.

For real-time adjustment, such as this, some value (or parameter) that is to be optimized is needed. The value (or parameter) can be optimized relative to a known value or relative to itself.

As an example, consider the cornea image. The scan profile could start with an approximate parabola, take a B-scan, identify the front surface of the cornea, fit a new curve to the front surface, modify the parabola and reacquire the B-scan with the new profile. This could be done one or more times based on the level of flatness required.

Alternatively, the front surface could be identified in a particular A-scan and the path length adjusted to keep the front surface at a given depth or within some range. The system could re-acquire the A-scan based on the new path length or the path length adjustment could be made prior to taking the A-scan of the next position in the scan. In this second case, the image would not be completely flat since adjustments would be made after a shift had occurred, but it may provide sufficient flattening to keep the sample with the image depth of the system.

As another example, consider retinal imaging in the periphery where the distance from the lens is sufficiently different to change the spot size on the retina. The focus could then be dynamically adjusted during the scan to minimize the spot size, using the signal strength as the optimization parameter. In this case, the goal is to maximize the signal (either overall or for a portion of the image) and so the optimization is a relative one since the signal strength at one value of the focus is compared to the signal strength at another value of the focus. Again, the system could optimize at a given location with multiple A-scans or over a sequence of A-scans or over one or more B-scans.

Dispersion compensation can also be optimized for cases where the dispersion varies as a function of depth. Since dispersion compensation occurs completely within the software processing of the image (unlike path length and focus adjustment, which change the physical setup of the system), this optimization can be done after the image is acquired. For example, a baseline dispersion compensation can be used to acquire the images and perhaps provide good enough signal to allow optimization of the path length and the focus, but as a post-processing step. The dispersion could then be optimized across a B-scan (or 3D volume) based on signal strength or some other parameter. The acquired raw image can be reprocessed multiple times to optimize the dispersion locally in different regions with the final combined optimized image presented to the user and saved.

### OTHER APPLICATIONS

Although this invention has been described primarily in terms of imaging the eye, there are numerous other applications where this system will have advantages. Eye imaging has focused on human eyes, but this system will work for other eyes as well, including human enfant and animals, including pigs, birds, rats, mice, rabbits, dogs, cats, horses, cows, and others. For curved scanning there may be variations on the scanning optics so that the size and working distance of the optics (lenses or mirrors or other) match the size and imaging range needed to cover the curvature of the sample.

There are numerous industrial and research applications that may need adjustable reference arm, focus and dispersion compensation over the lateral scanning range of the OCT system. Some materials that may be imaged include glass, plastic and other materials and combinations thereof. Some possible sample items include glass pipets, plastic tubing, and other samples.

It may be advantageous to use this invention in combination with either the high resolution or ultra-deep imaging OCT spectrometers and systems that Wasatch Photonics designs and builds. High resolution systems have spectrometers that typically cover more than 80 nanometers around 840 nm. Wasatch Photonics build spectrometers with bandwidths of 160 nm, 200 nm and 300 nm centered from 800 nm to 850 nm. When used in combination with very broadband light sources, the axial resolution may range from 3 microns down to 1 micron or less. The broadband nature of the system typically means that the imaging depth is reduced. With reduced imaging depth, it may be more advantageous to have a system that can adjust the imaging depth to match the range of a sample, particularly a curved one. For example, if one wants to do very high resolution (1 to 2 micron) of a LASIK flap on the front of the cornea, the imaging depth may only be 1 to 2 millimeters, requiring adjustment to keep the front of the cornea and the entire flap in the imaging depth of the OCT system across the lateral scan.

Ultra-deep systems typically have an imaging depth of more than 4 millimeters; Wasatch Photonics has built spectrometer with imaging depths of 8.8 millimeters and 11 millimeters. Combined with adjustable reference, focus and dispersion, these systems may be able to measure samples with imaging ranges of several centimeters.

The claims are not limited to the specific implementations described above. Various modifications, changes and variations may be made in the arrangement, operation and details of the implementations described herein without departing from the scope of the claims.

What is claimed is:

1. An optical coherence tomography (OCT) imaging system that implements at least one of the following:
adjusting the path length of a reference arm during an OCT scan of a curved sample or between OCT scans;
adjusting the focus of a scanning beam as the scanning beam moves across the curved sample during the OCT scan or between OCT scans;
adjusting polarization control during the OCT scan or between OCT scans; and/or
changing dispersion compensation across the OCT scan or between OCT scans.

2. The OCT imaging system of claim 1, wherein the path length of the reference arm is adjusted during the OCT scan in order to keep the curved sample within the imaging depth of the OCT imaging system.

3. The OCT imaging system of claim 1, wherein:
the reference arm comprises a first focusing lens, a mirror, a first motorized translation stage, and a fiber connector; and
the first motorized translation stage moves the first focusing lens and the mirror relative to the fiber connector.

4. The OCT imaging system of claim 3, wherein the reference arm also comprises:
a second focusing lens; and
a second motorized translation stage that moves the second focusing lens relative to the mirror.

5. The OCT imaging system of claim 4, wherein:
the reference arm is implemented in an OCT engine;
the OCT engine comprises a controller card that controls the first motorized translation stage and the second motorized translation stage; and
the OCT imaging system comprises a computer that controls the controller card.

6. The OCT imaging system of claim 4, wherein the OCT imaging system comprises a computer that directly controls the first motorized translation stage and the second motorized translation stage.

7. The OCT imaging system of claim 1, wherein the reference arm comprises a rapid scanning optical delay.

8. The OCT imaging system of claim 1, wherein:
the OCT imaging system comprises an OCT scan head;
the OCT scan head comprises a liquid lens; and
the liquid lens allows the focal point of the scanning beam to be varied in depth as the scanning beam is scanned laterally.

9. The OCT imaging system of claim 8, wherein:
the OCT scan head also comprises a fiber optic cable, a connector, one or more collimating lenses, one or more scanning mirrors, and one or more focusing lenses;
light enters the OCT scan head via the fiber optic cable;
the light exits the fiber optic cable at the connector and is collimated by the one or more collimating lenses;
collimated light exits the one or more collimating lenses and passes through the liquid lens;
the liquid lens imparts focusing or defocusing to the collimated light;
focus-adjusted light exits the liquid lens and is scanned laterally by the one or more scanning mirrors; and
the one or more focusing lenses focus the scanning beam on the curved sample.

10. The OCT imaging system of claim 1, wherein the OCT imaging system comprises an OCT scan head, and wherein the OCT scan head comprises:
a fiber optic cable;
a connector;
at least one collimating lens; and
a motorized translation stage that moves the at least one collimating lens relative to the fiber optic cable and the connector.

11. The OCT imaging system of claim 10, wherein:
the OCT scan head also comprises one or more scanning mirrors and one or more focusing mirrors;
light enters the OCT scan head via the fiber optic cable;
the light exits the fiber optic cable at the connector;
the movement of the at least one collimating lens relative to the fiber optic cable and the connector causes focus-adjusted light to exit the at least one collimating lens;
the one or more scanning mirrors move the focus-adjusted light laterally; and
the one or more focusing mirrors focus the scanning beam on the curved sample.

12. The OCT imaging system of claim 1, wherein changing the dispersion compensation across the OCT scan comprises changing the dispersion compensation as the scanning beam moves laterally across the curved sample.

13. The OCT imaging system of claim 1, wherein changing the dispersion compensation across the OCT scan comprises:
acquiring a raw OCT image using a baseline dispersion compensation; and
optimizing the dispersion compensation as a post-processing step; and
optimizing the dispersion compensation locally within a range of A-scans, B-scans, or C-scans and combining such locally optimized regions to a final image presented to the user.

14. The OCT imaging system of claim 1, wherein:
performing dispersion compensation comprises introducing correction terms after wavelength data is re-sampled to wavenumber; and
changing the dispersion compensation comprises adjusting the correction terms.

15. The OCT imaging system of claim 1, wherein:
the OCT imaging system adjusts the path length of the reference arm, adjusts the focus of the scanning beam, adjusts the polarization control and/or changes the dispersion compensation based on a pre-set scan profile; and
the pre-set scan profile is static during the OCT scan.

16. The OCT imaging system of claim 1, wherein:
the OCT imaging system adjusts the path length of the reference arm, adjusts the focus of the scanning beam, adjusts the polarization control and/or changes the dispersion compensation based on a profile; and
the profile is modified during the OCT scan or between OCT scans.

17. The OCT imaging system of claim 16, wherein modifying the profile comprises optimizing a parameter relative to a known value or relative to itself.

18. The OCT imaging system of claim 1, wherein:
the OCT imaging system comprises a first polarization controller in the reference arm and a second polarization controller in a sample arm;
the first polarization controller is optimized at system setup or startup; and
the second polarization controller is adjusted to compensate for changes in the curved sample or in the sample arm.

19. The OCT imaging system of claim 1, wherein:
the reference arm comprises a liquid lens; and
the focal length of the liquid lens is changed in order to implement optical power control.
